SEQUENCE LISTING (8 sequences)

SEQ ID NO: 1 - protein, 183 aa, Hepatitis B virus
MDIDPYKEFGATVELLSFLPSDFFPSVRDLLDTAAALYRDALESPEHCSPHHTALRQAILCWGDLMTLATWVGTNLEDPASRDLVVSYVNTNVGLKFRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 2 - DNA, 549 bp, Hepatitis B virus
  source 1..549: mol_type = other DNA
atggacattgacccttataaagaatttggagctactgtggagttactctcgtttttgccttctgacttctttccttccgtacgagatcttcttgataccgccgcagctctgtatcgggatgcattagagtctcctgagcactgcagccctcaccatactgccttaaggcaagcaattctttgctggggagacttaatgactctagctacctgggtgggtactaatttagaagatccagcatctagggacctagtagtcagttatgtcaacactaatgtgggcctaaagttcagacaattattgtggtttcacatttcttgtctcacttttggaagagaaacggttctagagtatttggtgtcttttggagtgtggattcgcactcctccagcttatagaccaccaaatgcccctatcctgtcgacgcttccggagactactgttgttcgtcgccgtggccgttccccgcgtcgccgtactccgtcgccgcgtcgccgtcgctctcaatcgccgcgtcgccgtcgttctcaatctcgtgaatctcaatgt

SEQ ID NO: 3 - protein, 149 aa, Hepatitis B virus
MDIDPYKEFGATVELLSFLPSDFFPSVRDLLDTAAALYRDALESPEHCSPHHTALRQAILCWGDLMTLATWVGTNLEDPASRDLVVSYVNTNVGLKFRQLLWFHISCLTFGRETVLEYLVSFGVWIRTPPAYRPPNAPILSTLPETTVV

SEQ ID NO: 4 - DNA, 447 bp, Hepatitis B virus
  source 1..447: mol_type = other DNA
atggacattgacccttataaagaatttggagctactgtggagttactctcgtttttgccttctgacttctttccttccgtacgagatcttcttgataccgccgcagctctgtatcgggatgcattagagtctcctgagcactgcagccctcaccatactgccttaaggcaagcaattctttgctggggagacttaatgactctagctacctgggtgggtactaatttagaagatccagcatctagggacctagtagtcagttatgtcaacactaatgtgggcctaaagttcagacaattattgtggtttcacatttcttgtctcacttttggaagagaaacggttctagagtatttggtgtcttttggagtgtggattcgcactcctccagcttatagaccaccaaatgcccctatcctgtcgacgcttccggagactactgttgtt

SEQ ID NO: 5 - protein, 142 aa, Hepatitis B virus
MDIDPYKEFGATVELLSFLPSDFFPSVRDLLDTAAALYRDALESPEHCSPHHTALRQAILCWGDLMTLATWVGTNLEDPASRDLVVSYVNTNVGLKFRQLLWFHISCLTFGRETVLEYLVSFGVWIRTPPAYRPPNAPILST

SEQ ID NO: 6 - DNA, 426 bp, Hepatitis B virus
  source 1..426: mol_type = other DNA
atggacattgacccttataaagaatttggagctactgtggagttactctcgtttttgccttctgacttctttccttccgtacgagatcttcttgataccgccgcagctctgtatcgggatgcattagagtctcctgagcactgcagccctcaccatactgccttaaggcaagcaattctttgctggggagacttaatgactctagctacctgggtgggtactaatttagaagatccagcatctagggacctagtagtcagttatgtcaacactaatgtgggcctaaagttcagacaattattgtggtttcacatttcttgtctcacttttggaagagaaacggttctagagtatttggtgtcttttggagtgtggattcgcactcctccagcttatagaccaccaaatgcccctatcctgtcgacg

SEQ ID NO: 7 - RNA, 31 nt, synthetic construct
  source 1..31: mol_type = other RNA
ccctatctaatcgacacttccggagactacg

SEQ ID NO: 8 - RNA, 28 nt, synthetic construct
  source 1..28: mol_type = other RNA
gtcgattagataggggcatttggtggtc

(19) United States
(12) Patent Application Publication
(10) Pub. No.: US 2026/0022351 A1
(43) Pub. Date: Jan. 22, 2026

(54) COMPOUNDS AND METHODS FOR CHEMICALLY TAGGING MACROMOLECULE CARGO INSIDE AND OUTSIDE OF VIRUS-LIKE PARTICLES

(71) Applicant: The Trustees of Indiana University, Bloomington, IN (US)

(72) Inventors: Adam Zlotnick, Bloomington, IN (US); Tariq Hussain, Bloomington, IN (US); Michael VanNieuwenhze, Bloomington, IN (US); Sicheng Chie, Bloomington, IN (US)

(21) Appl. No.: 19/276,586

(22) Filed: Jul. 22, 2025

Related U.S. Application Data
(60) Provisional application No. 63/674,106, filed on Jul. 22, 2024.

(51) Int. Cl. C12N 7/00; C07K 1/107; C07K 14/005

(52) U.S. Cl. CPC C12N 7/00; C07K 1/1077; C07K 14/005; C12N 2710/00022; C12N 2710/00023; C12N 2710/00051

(57) ABSTRACT

Disclosed herein is a novel tool to associate protein cargo molecules to the inside and/or outside of a Hepatitis B Virus (HBV) virus-like particle (VLP). The novel tool being composed of a sulfamoylbenzamide (SBA) antiviral, linker, and protein.

### CROSS REFERENCE TO RELATED APPLICATIONS

This application claims the benefit under 35 U.S.C. § 119(e) of U.S. Provisional Application No. 63/674,106 entitled “COMPOUNDS AND METHODS FOR CHEMICALLY TAGGING MACROMOLECULE CARGO INSIDE AND OUTSIDE OF VIRUS-LIKE PARTICLES”, filed on Jul. 22, 2024, which is incorporated by reference in its entirety.

### STATEMENT OF GOVERNMENTAL RIGHTS

This invention was made with government support under AI144022 awarded by National Institutes of Health. The Government has certain rights in the invention.

### INCORPORATION OF SEQUENCE LISTING

A paper copy of the Sequence Listing and a computer readable form of the sequence containing the file named “IU-2025-007-02-US_st26.xml”, prepared on Jul. 22, 2025, which is 9.75 KB in size, are provided herein and are herein incorporated by reference.

### FIELD OF THE INVENTION

The present disclosure relates generally to the use of Hepatitis B Virus (HBV) as a delivery mechanism for cargo molecules, such as in vaccine development and drug delivery.

### BACKGROUND

HBV is a hepatotropic, partially double-stranded DNA virus, belonging to the Hepadnaviridae family. The HBV capsid, a complex of 120 homodimeric capsid proteins (Cp) arranged with T=4 icosahedral symmetry, has been used as a platform for vaccine development. HBV Cp is comprised of two domains: an N-terminal assembly-active domain (residues 1-149) and C-terminal genome packaging domain (residues 150-183). The assembly domain is sufficient to produce regular capsids in vitro and is readily expressed and purified with high yield from E. coli (e.g., 200 mg of Cp per L of growth media). Purified protein is readily assembled in vitro. For use as vaccine, several groups have inserted epitopes into a loop exposed on the capsid surface; such modifications do not always work and require re-optimization of expression.

Several groups have worked on methods to package protein cargo inside an HBV virus-like particle (VLP). This requires a method to overcome the high concentration gradient with many copies of cargo inside an enclosed volume versus a dilute solution of cargo in solution. These strategies include leveraging native or non-native interactions between protein cargo molecules and interior surface of VLP. For example, in a 2008 study by Lee and Tan, capturing Green Fluorescent Protein (GFP) molecules by disassembly and reassembly of dimeric Cp. While the study was successful in packaging GFP inside an HBV VLP, it was incredibly inefficient and required the presence of high concentrations of cargo. Another group capitalized on the positively charge N-terminus of the capsid protein of cowpea chlorotic mottle virus for charge-mediated encapsulation of enzymes. Here, they tagged enzyme cargos with nucleic acid and demonstrated the packaged enzyme cascade systems were active. While this strategy is simple it requires complex modification of the cargo. Several groups have shown genetic fusion of protein cargo to P22 scaffold proteins spontaneously co-assembles with P22 capsid proteins to achieve high loading densities of cargo. This approach also requires significant modifications to cargo and scaffold proteins.

Delivery of protein and ribonucleoprotein complexes are an attractive means to address many genetic deficiencies though has been bottlenecked due to their size, hydrophilicity, and susceptibility to degradation. VLPs provide an ideal container to package therapeutic proteins, and deliver those protein molecules to a cell. However, to be a relevant clinical tool, effective strategies to package intact therapeutic proteins inside the VLP and decorating the exterior for cell-specific targeting or receptor binding are needed. The present disclosure describes methods and tools to overcome the limitations and challenges described above to package cargo inside a VLP.

### SUMMARY OF THE INVENTION

A first aspect of the invention includes a tripartite tool for attaching a protein cargo of interest to the inside and outside of an HBV virus-like particle (VLP), composed of a capsid assembly modulator (CAM), a linker, and a protein of interest, wherein the CAM is attached to the linker and the protein of interest is attached at the opposite end of the linker.

A second aspect of the invention includes a tripartite tool for attaching a protein cargo of interest to the inside and outside of an HBV virus-like particle (VLP) wherein the CAM is sulfamoylbenzamide (SBA).

A third aspect of the invention includes a tripartite tool for attaching a protein cargo of interest to the inside and outside of an HBV virus-like particle (VLP) wherein the length of the linker extends from the capsid wall to the capsid lumen

A fourth aspect of the invention includes a tripartite tool for attaching a protein cargo of interest to the inside and outside of an HBV virus-like particle (VLP) wherein the linker is terminated with a maleimide

A fifth aspect of the invention includes a tripartite tool for attaching a protein cargo of interest to the inside and outside of an HBV virus-like particle (VLP) wherein the SBA is chemically attached to the linker.

A sixth aspect of the invention includes a tripartite tool for attaching a protein cargo of interest to the inside and outside of an HBV virus-like particle (VLP) wherein the protein of interest contains a single reactive cysteine.

A seventh aspect of the invention includes a tripartite tool for attaching a protein cargo of interest to the inside and outside of an HBV virus-like particle (VLP) wherein the protein if interest is attached to the linker through a reaction of the maleimide of the linker and the reactive cystine of the protein of interest.

An eight aspect of the invention includes a method of attaching a protein cargo to the exterior of an HBV virus-like particle (VLP), comprising the steps of preparing the tripartite tool for attaching a protein cargo of interest to the inside and outside of an HBV virus-like particle (VLP), and mixing the tripartite tool with pre-assembled HBV VLP capsids.

A ninth aspect of the invention includes a method of attaching a protein cargo of interest to the interior of an HBV virus-like particle (VLP), comprising the steps of preparing the tripartite tool for attaching a protein cargo of interest to the inside and outside of an HBV virus-like particle (VLP), mixing the tripartite tool with HBV capsid protein (CP) homodimer, and inducing assembly of the HBV VLP capsids.

A tenth aspect of the invention includes a method of attaching a protein cargo of interest to the interior of an HBV virus-like particle (VLP) further comprising the step of removing any protein of interest present on the exterior of the HBV VLP capsids by treating the assembled HBV VLP capsids with a reducing agent.

An eleventh aspect of the invention includes a tripartite tool, wherein the CAM is a sulfamoylbenzamide (SBA) of formula (I)

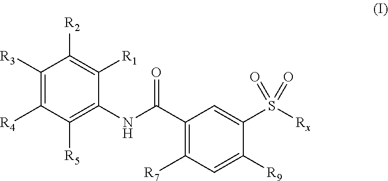

including hydrates, solvates, pharmaceutically acceptable salts, prodrugs and complexes thereof, wherein:
Rₓ is a formula of (I-1), (I-2), or (I-3)

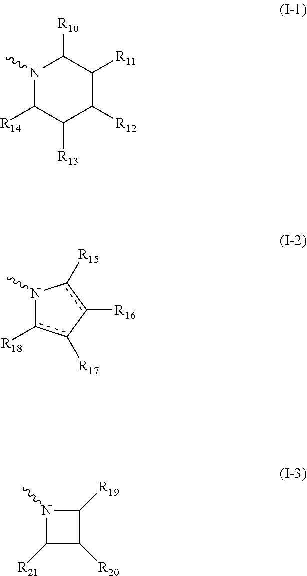

the wave-line denoting the connection to Formula I;
wherein
R₁ is hydrogen;
R₂ is selected from a group consisting of hydrogen, methyl, trifluoromethyl, fluorine, and chlorine;
R₃ is selected from a group consisting of hydrogen, methyl, fluorine, and chlorine;
R₄ is selected from a group consisting of hydrogen, fluorine, chlorine, and methyl;
R₅ is selected from a group consisting of hydrogen and chlorine;
R₇ is selected from a group consisting of hydrogen, chlorine, fluorine, and bromine;
R₉ is selected from a group consisting of hydrogen, methyl, fluorine, and chlorine; and
wherein when Rx is a formula of (I-1), one of R₁₀, R₁₁, R₁₂, R₁₃, and R₁₄ is OH and the remaining of R₁₀, R₁₁, R₁₂, R₁₃, and R₁₄ are each hydrogen;
when Rx is a formula of (I-2), one of R₁₅, R₁₆, R₁₇, and R₁₈ is OH, and the remaining of R₁₅, R₁₆, R₁₇, and R₁₈ are each hydrogen;
when Rx is a formula of (I-3), one of R₁₉, R₂₀, and R₂₁ is OH, and the remaining of R₁₉, R₂₀, and R₂₁ are each hydrogen;
[symbol] markings represent single or double bonds.

A twelfth aspect of the invention includes a tripartite tool, wherein the CAM is a sulfamoylbenzamide (SBA) of formula (I) and wherein R₂, R₃, and R₄ are each fluorine.

A thirteenth aspect of the invention includes a tripartite tool, wherein the CAM is a sulfamoylbenzamide (SBA) of formula (I) and wherein R₁ and R₅ are each hydrogen.

A fourteenth aspect of the invention includes a tripartite tool, wherein the CAM is a sulfamoylbenzamide (SBA) of formula (I) and wherein R₇ is hydrogen and R₉ is fluorine.

A fifteenth aspect of the invention includes a tripartite tool, wherein the CAM is a sulfamoylbenzamide (SBA) of formula (I) and wherein Rₓ is

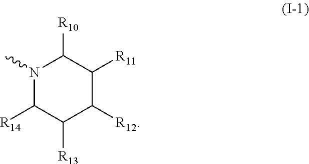

A sixteenth aspect of the invention includes a tripartite tool wherein Rₓ is (I-1) and R₁₀, R₁₁, R₁₃, and R₁₄ are each hydrogen and R₁₂ is OH.

A seventeenth aspect of the invention includes a tripartite tool, wherein the CAM is a sulfamoylbenzamide (SBA) of formula (I) and wherein Rₓ is

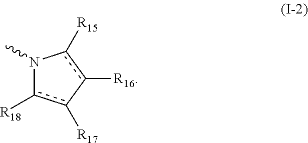

An eighteenth aspect of the invention includes a tripartite tool wherein Rₓ is (I-2) and one of R₁₆ and R₁₇ is OH and the other is hydrogen.

A nineteenth aspect of the invention include a tripartite tool wherein Rₓ is (I-2) and wherein [symbol] markings represent single bonds.

A twentieth aspect of the invention includes a tripartite tool wherein the CAM is a sulfamoylbenzamide (SBA) of formula (I) and wherein Rₓ is

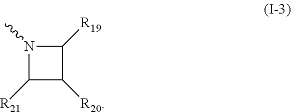

A twenty-first aspect of the invention includes a tripartite tool wherein Rₓ is (I-3) and wherein R₁₉ and R₂₁ are each hydrogen and R₂₀ is OH.

A twenty-second aspect of the invention includes a tripartite tool wherein the CAM is a sulfamoylbenzamide (SBA) of formula (I) and wherein Rx is one of

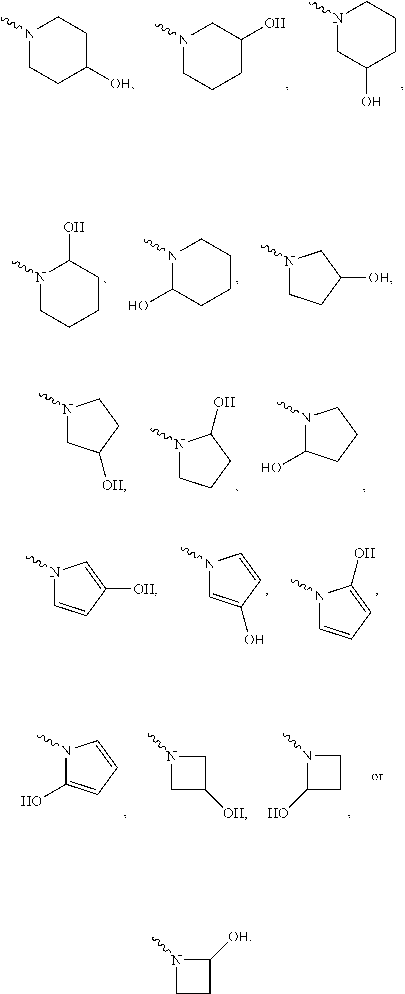

A twenty-third aspect of the invention includes a tripartite tool wherein the compound of Formula (I) is

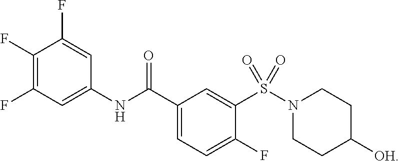 [SMILES: O=C(Nc1cc(F)c(F)c(F)c1)c1ccc(F)c(S(=O)(=O)N2CCC(O)CC2)c1]

A twenty-fourth aspect of the invention includes a tripartite tool, wherein the linker is

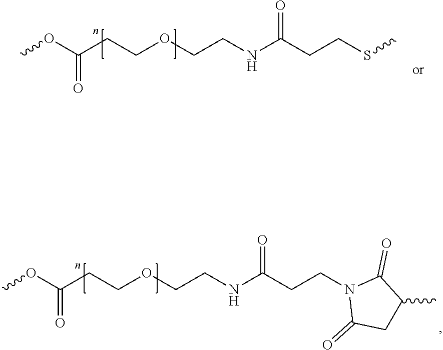

the wave-line from the sulfur or carbon atom denoting the connection to the protein, and the wave line from the oxygen atom denoting the connection to a compound of Formula (I);
wherein n is an integer from 1 to 20.

A twenty-fifth aspect of the invention includes a tripartite tool wherein the tripartite tool is

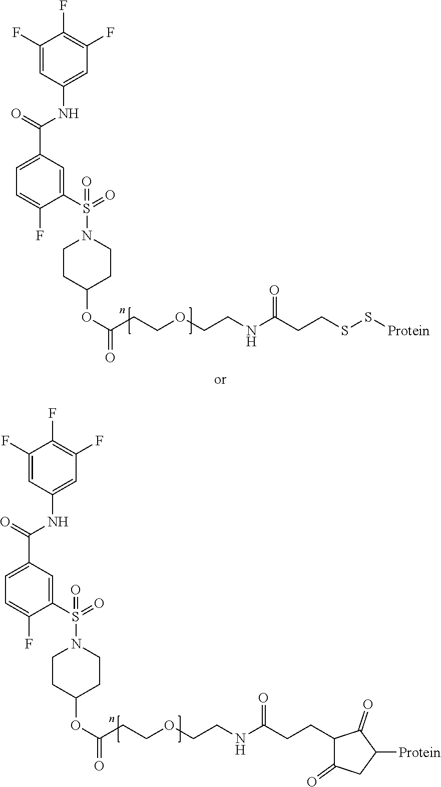

wherein n is an integer from 1 to 20.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A is an illustration of one embodiment of the invention where the size of the capsid protein is modified in conjunction with the use of the tripartite construct to improve cargo packaging.

FIG. 1B is an illustration of one embodiment of the invention where isotonic concentration is increased in conjunction with the use of the tripartite construct to improve cargo packaging.

FIG. 2 is a graphical demonstration of the tripartite tool's ability to drive HBV assembly.

FIG. 3A-3D are graphs comparing the tripartite tool using HAP and SBA in external decoration of pre-assembled HBV VLPs (FIG. 3A and 3C) and in co-assembly reactions (FIG. 3B and 3D).

FIG. 4 shows transmission electron microscopy images of capsid assembly using embodiments CAMs in the tripartite tool—HAP and SBA.

FIG. 5A-5B: show the particle surface charge and particle volume to evaluate location of SBA-GFPs on or within the VLPs.

FIG. 6A-6B show performance/activity evaluation of SBA-GFP

FIG. 7 shows images of negative-stain TEM of purified VLPs, with capsid control (left) and capsids co-assembled with 20 μM SBA-GFP (right).

FIG. 8A-8B are chromatographs illustrating the capsid peak for SBA-GFP and Cp149 co-assembly before (FIG. 8A) and after addition of BME (FIG. 8B).

FIG. 8C-8D are chromatographs illustrating SBA-GFP binding to preassembled Cp149 capsids before (FIG. 8C) and after addition of BME (FIG. 8D).

FIG. 8E-8F display the mass distribution, determined by mass photometry, of purified coassembled VLPs without and with SBA-GFP before (FIG. 8E) and after (FIG. 8F) β-mercaptoethanol treatment.

FIG. 9A shows protein modeling simulations indicating Cp149 linker can crowd quasi-sixfold pore exit-way. Quasi-sixfold pore topology defined by HOLE considering residues 1-149 versus residues 1-142. In narrow Cp149 hexamers, HOLE fails to trace a pathway due to linker residues crowding and occluding the pore exit-way. When residues 143-149 (red) are omitted, HOLE detects the unconcluded pore opening.

FIG. 9B shows a Representative conformer of a heavily crowded exit-way, highlighting only the Cα atoms of offending linker residues.

FIG. 9C is a graphical illustration of the percentage of hexamer conformers (out of 1.5 million) in which the pore exit-way is crowded by increasing numbers of linker residues (represented by their Cα atoms). Pore exit-way modeled as a cylinder extending through and below the pore opening (inset), using either the pore diameter determined by HOLE (black, likely an underestimation of the exit-way funnel) or the pore diameter defined by the distance between Pro135-Asn136 pairs across the hexamer (gray). The former suggests as many as 11 residues simultaneously crowding the exit-way, while the latter suggests as many as 17. Percentages indicated above each bar.

FIG. 10A depicts blocking of an internal 7-residue c-terminal linker during co-assembly blocks accumulation of SBA-GFP cargo inside the VLP.

FIGS. 10B shows mass photometry of Cp142 (blue) and Cp149 (black) VLPs. The mass distributions of the purified VLPs show that Cp142 VLPs preference T=3 geometry while Cp149 VLPs prefer T=4 geometry. The theoretical masses of Cp142 T=3 and T=4 particles are 2.79 MDa and 3.85 MDa (based on a 32,060 Da dimer), respectively. The mass predictions are within error. The inset shows a representative electron microscopy micrograph of purified VLP, where the T=4 are labeled with a yellow asterisk.

FIGS. 10C and 10D are chromatographs of Cp142 dimers co-assembled with SBA-GFP pre-BME (C) and post-BME (D) treatment. Only the capsid peak are shown. Absorbance at 280 nm (solid lines) was monitored for elution of total protein, absorbance at 488 nm (dotted lines) was monitored to track SBA-GFP elution, and the number of SBA-GFP per VLP (dashed lines).

FIG. 10E and 10F are chromatographs of Cp142 dimers co-assembled with SBA-GFP clusters pre-BME (E) and post-BME (F) treatment. SBA-GFP binding was concentration dependent and higher stoichiometries typically led to assembly of more T=4 VLPs. Cp142 dimers packaged significantly more SBA-GFPs. Only the capsid peak are shown. Absorbance at 280 nm (solid lines) was monitored for elution of total protein, absorbance at 488 nm (dotted lines) was monitored to track SBA-GFP elution, and the number of SBA-GFP per VLP (dashed lines).

### DETAILED DESCRIPTION

Disclosed is a novel tool to associate protein cargo molecules to the inside and outside of a Hepatitis B Virus (HBV) virus-like particle (VLP). There are advantages to delivering a protein cargo to target cell as a therapeutic, rather than a DNA or RNA. However, a person of skill in the art understands that packaging active and folded proteins inside VLPs is not trivial. It requires overcoming a steep entropic gradient which can be addressed by covalent methods, but these may cause misfolding of cargo or capsid proteins and may inhibit assembly of the VLP. To enable modular encapsulation of protein cargo, we showcase a chemical biology approach for directing protein cargo inside using simple but effective strategies that can be applied to similar VLP systems.

This tool allows for high loading densities of cargo proteins inside a VLP without the need for modifying the viral protein and requiring only a single reactive site on the cargo protein. Cargo molecule copy number and placement (inside or outside the VLP) can be controlled during assembly reactions. This invention presents a unique means of packaging a macromolecular cargo and provides targeted VLP delivery systems.

The tripartite tool may be composed of a Capsid Assembly Modulator (CAM), a linker (L), and a protein (P).

### Capsid Assembly Modulators (CAM)

CAMs are assembly agonists that as therapeutics drive assembly of empty, defective viruses. The CAM may include a sulfamoylbenzamide (SBA) of general formula (I).

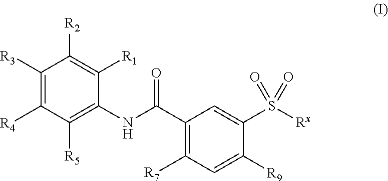

including hydrates, solvates, pharmaceutically acceptable salts, prodrugs and complexes thereof, wherein:
R1 is hydrogen;
R2 is selected from a group consisting of hydrogen, methyl, trifluoromethyl, fluorine, and chlorine;
R3 is selected from a group consisting of hydrogen, methyl, fluorine, and chlorine;
R4 is selected from a group consisting of hydrogen, fluorine, chlorine, and methyl;
R5 is selected from a group consisting of hydrogen and chlorine;
R7 is selected from a group consisting of hydrogen, chlorine, fluorine, and bromine;
R9 is selected from a group consisting of hydrogen, methyl, fluorine, and chlorine;
Rx is

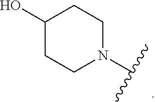 [SMILES: *N1CCC(O)CC1]

For example, the SAB may include NVR3-778 (also known as SBA_R01) represented by formula (II)

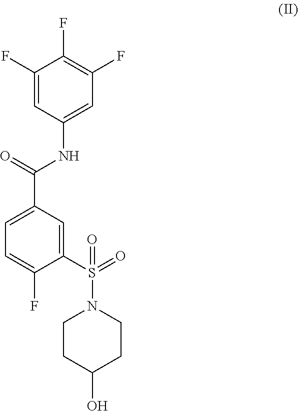 [SMILES: O=C(Nc1cc(F)c(F)c(F)c1)c1ccc(F)c(S(=O)(=O)N2CCC(O)CC2)c1]

CAMs bind to a hidden but accessible pocket at the dimer-dimer interface. Mechanistically, all CAMs accelerate assembly but differ in their products depending on the chemotype. CAMs favor Cp to adopt as assembly active state and stabilize subunit interactions (adds to the buried hydrophobic surface area between dimers), supporting errors during the assembly process. In vivo, CAMs deplete the cell of newly translated Cp that otherwise would have been used to make new virus. CAMs may generally be classified into two groups, CAM-A and CAM-E. Assembly in the presence of CAM-As, such as heteroaryldihydropyrimidines (HAPs), may produce large aberrant structures. CAM-Es, sulfamoylbenzamides (SBAs) drive assembly of empty capsids, thus inhibiting the formation of infectious DNA containing particles.

Sulfamoyl benzamides (SBAs) are one group of CAMs notable for their ability to drive assembly of morphologically normal empty capsids. Based on structure, the SBA can be modified without impeding its activity. The CAM, such as an SBA, is chemically attached to a linker predicted to extend from the capsid wall to the capsid lumen.

### Proteins for Packaging (Cargo Proteins)

The protein P may include any protein of interest for attaching to the inside and outside of an HBV virus-like particle (VLP). As used herein, the protein of the tripartite tool may also be referred to as “cargo” or “cargo proteins.” These terms are used interchangeably in this specification.

There is sufficient room within the VLP capsid for up to 3 MDa of payload, which typically would contain the mass of the viral genome and polymerase. However, large proteins of >50 kDa may be limited to one per quasi-sixfold; that is 30 per T=4 capsid (for a 50 kDa protein that equates to 1.5 MDa). Examples of packaged protein or protein-nucleic acid complexes which may be packed via the tripartite tool disclosed herein include CRISPR associated proteins with bound guide RNA, including CAS9, CAS12a. CAS12e, or nucleases such as TALENs. Other exemplary proteins include immunostimulants, such as interferon alpha, gamma, or lambda; or signaling proteins such as tyrosine kinases or VEGF; or metabolic proteins such frataxin. Further exemplary cargos include anticancer proteins such as p53 or cytokines like IL2. Additionally, a person of skill in the art would appreciate, that many of the proteins used in gene therapy could be better delivered as proteins rather than genes and the present tripartite tool creates a viable option for delivering these therapies as proteins.

As a model cargo we use a Green fluorescent protein (GFP) with a single reactive cysteine. As disclosed herein, using the tripartite tool, we can attach this cargo to the exterior of the capsid by allowing it to react with pre-assembled capsids. We can also, preferentially attach the cargo to the interior by mixing the tripartite tool with CP homodimer and then inducing assembly under conditions where we find most of the cargo is on the capsid interior.

### Linkers

The linker may include phosphate-containing groups (e.g., phosphoserine, phosphotyrosine), bisphosphonates, carboxylate-functionalized moieties (e.g., poly (aspartic acid)), catechol groups, and silane-based linkers for surface modification. Additional linkers for use in the tripartite tool include longer alkyl chains. For example, the linker may include substituted or unsubstituted, straight or branched alkyl chains having 6 to 100 total carbon atoms in the chain, or may have any number of carbon atoms encompassed within that range. In some embodiments, the linker may include polyethylene glycol (PEG) or other hydrophilic polymers. Optionally, the linker may include cleavable elements such as enzyme-sensitive peptide sequences, pH-labile bonds, redox-sensitive disulfide bridges, DNA/RNA, or labile chains that release cargo in response to cleavage by reducing agent or nuclease.

For example, a PEG containing linker may include n ethylene glycol repeat units. For example, n may be an integer from 1 to 50. For example, n may be 1, 2, 3, 4, 5, 6, 7, 8, 9, 10, 11, 12, 13, 14, 15, 16, 17, 18, 19 or 20.

In a preferred embodiment, the linker is terminated with a maleimide, reactive with cysteine thiol. In another preferred embodiment, the linker is pegylated succinimidyl 3-(2-pyridyldithio)propionate (SPDP) bifunctional crosslinker.

### Tripartite Constructs

The tripartite construct disclosed herein may have formula (III)

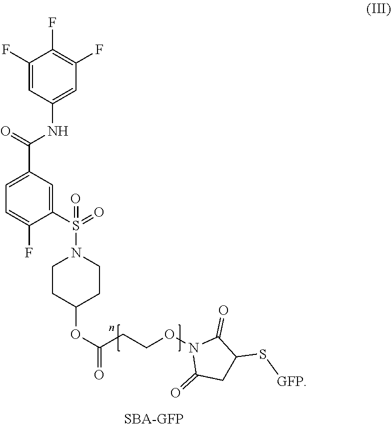

wherein n is an interger from 1 to 20. For example, n may be 1, 2, 3, 4, 5, 6, 7, 8, 9, 10, 11, 12, 13, 14, 15, 16, 17, 18, 19 or 20

Alternatively, the tripartite construct may have formula (VI)

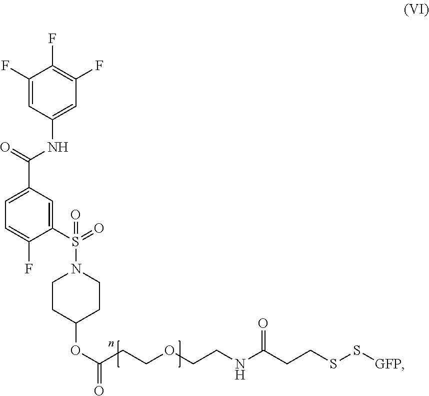

wherein n is an integer from 1 to 20. For example, n may be 1, 2, 3, 4, 5, 6, 7, 8, 9, 10, 11, 12, 13, 14, 15, 16, 17, 18, 19 or 20.

The tripartite construct tool may be used for attaching a protein cargo of interest to the outside or to the inside and the outside of an HBV virus-like particle (VLP). The tool has the capability to be mixed with Hepatitis B Virus (HBV) capsid proteins (Cp), and spontaneously package high densities of protein cargos inside the VLP. Additionally, this invention does not disturb the integrity or structure of the VLP container. Such an invention provides a straightforward means to achieving a highly functional VLP. The result is a generalizable tool to associate any cargo.

This invention provides novel tool towards developing and improving VLP technologies. VLPs offer many advantages to deliver protein therapeutics. VLPs are stable and uniform super-structures. Since VLPs are comprised of only the virus capsid proteins, they are non-infectious. VLPs tend to be extremely stable. The contents of a VLP are protected from immune surveillance and from attack by external enzymes. Because of these favorable characteristics, VLPs are often used as nanoreactors, vaccine carriers, or as imaging agents. Nonetheless, VLP utility has been hampered by lack of strategies to efficiently and specifically package therapeutic cargos of interest inside a VLP (and decorate its exterior surface).

An SBA-linker-cargo requires minimal modification, a functional handle (e.g, an accessible cysteine), and no modification to the VLP platform. This tool is generalizable to all type's cargos (RNA aptamers, genes, proteins, etc) as long it meets the above requirement. Since protein cargos are tagged with SBAs, it directs and associates the cargo to the HBV VLP and can be tuned by input concentration. Using SBA-linker-cargos external decorations can be purified away by using reversible linkers.

### Virus Capsids

Virus capsids are composed of one or many repeating capsid protein subunits that self-assembles around and packages their viral genome. Capsids that lack viral gene materials, or virus-like particles (VLPs), provide powerful platforms for studying catalysis, vaccines and immunotherapy development, and as super-fluorescent imaging agents. VLPs have also been used as carriers to deliver small molecules, nanoparticles, and gene transcripts, making them enticing nanocontainers for targeted delivery of protein therapeutics and ribonucleoprotein complexes.

Hepatitis B virus (HBV) is an enveloped double-stranded DNA virus that has an icosahedral capsid core. Upon self-assembly, the capsid or core protein (Cp) surrounds a pre-genomic RNA-polymerase intermediate that is reverse transcribed to double stranded DNA during virus maturation. The HBV Cp has been predominantly used as a platform to display large surface antigens for vaccination. Full length Cp is 183 residues (Cp183) (SEQ ID NO:1), though assembly studies have focused on Cp149 (SEQ ID NO:3), the assembly domain which lacks the 34 C-terminal residues of Cp183 which are part of a genome packaging that is dispensable for assembly of icosahedral capsids. Cp149 exists as a dimer that is mostly alpha-helical and is held together by a four-helix bundle that decorates the capsid exterior with spikes. Cp dimers assemble in vitro by increasing the ionic strength of the solution. Assembly is driven by buried hydrophobic surface and weak dimer-dimer interactions that make four contacts with neighboring dimers. HBV assembly has sigmodal kinetics; a slow rate-limiting nucleation and fast elongation step where free dimers stochastically add on to the growing capsid lattice resulting in a 120-dimer T=4 (95%) and 90-dimer T=3 (5%). The ratio of T=4 and T=3 particles are dependent on the length of its C-terminus, however the predominant T=4 capsid has a diameter of 36 nm with a 25 nm interior to package 3.2 kbp equating to a striking mass of nearly 2 MDa. As a VLP delivery platform, the HBV capsid strengths are in its in vitro self-assembly into well-defined particles, tolerance to large modifications of its 240 monomeric subunits (especially surface spike modifications), and overexpression in bacterial cell culture. HBV Cp molecules suitable for packaging using the tripartite tool disclosed herein include full length Cp183 as well as the truncated Cp molecules, including Cp149 (SEQ ID NO:3) and Cp142 (SEQ ID NO:5),

### Methods of Cargo Packaging Using Tripartite Tool

Maximally, several hundred protein molecules can fit into an HBV VLP. Though several considerations must be made including charge, orientation, packing, and VLP curvature, etc. Realistically, a VLP can achieve packaging from a few to several dozen protein molecules the size of GFP molecules. With this approach, we achieved 25-50% of this upper limit before making any major CAM optimizations.

In one embodiment, the tripartite tool having an having an SBA CAM is utilized in the packaging. SBAs are known to B and C sites on the capsid. There are 60 pockets per site located around the quasi-sixfold pores (30 per T=4). In total 120 sites are available yet roughly 25% of sites are filled with SBA-GFP. The inability for SBA-GFPs to saturate the capsid is unknown, though one could speculate that it is likely a steric issue. Four sites are available per quasi-sixfold on a T=4 VLP, though only one to two bulky SBA-GFP molecules can fit preventing full occupancy. Alternatively, SBAs may bind by a cooperative mechanism and may be limited by the opening of free binding sites. Nonetheless, this feature provides an opportunity for multivalent display of CAM-cargos. Where HAPs and SBAs can be used synergistically to decorate the VLP for two unique functionalities.

In another embodiment, tripartite tool comprising HAP and tripartite tool comprising SBA are used together. The intrinsic behaviors of HAPs and SBAs opens the door for dual display strategies. For instance, one cargo could be packaged via SBA and Cp co-assembly, while the HAP can externally decorate the resulting VLPs. This provides new avenues for developing multi-functional VLPs, a valuable addition to address complex delivery challenges particularly in the context of cancer therapy. The SBA-GFP system is practical for cargo generalizability, requiring only minimal modification to the cargo protein. In this case, we engineer a solvent accessible cysteine; a simple addition to any cargo protein.

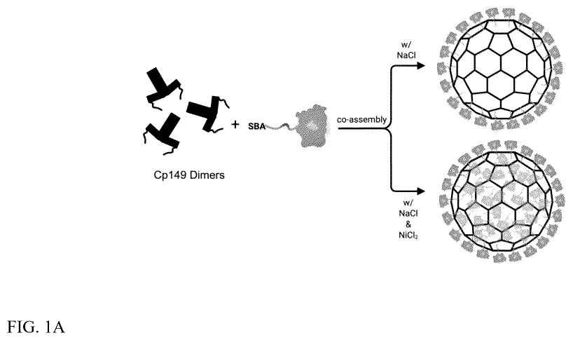

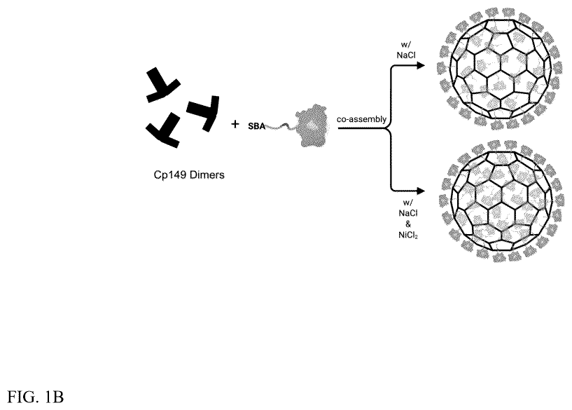

In another embodiment, the ionic strength of the buffer was increased during packaging. The SBA-GFP could be packaged by redirecting the cargo into the capsid during assembly by adding NiCl₂ to assembly buffer. (FIG. 1B)

In another embodiment, the tripartite tool is utilized in conjunction with a modified core protein. (FIG. 1A). Interestingly, we also found that truncating seven residues off the Cp149 C-terminal linker domain, called Cp142, promoted encapsidation of SBA-GFPs without the need for nickel ions. First, favoring of T=4 capsid polymorphs suggest multiple copies of GFP are packaged and favor a VLP with more space. This is in agreement with the relationship between cargo size and capsid assembly product. Second, this suggests the linker domain on Cp149 may interact with or near the CAM binding pocket and likely is to have some biological role. This is in agreement with several studies showing full-length HBV Cp, Cp183, C-terminal domain protruding from the two-fold (quasi six-folds) pores.⁷⁹⁻⁸⁰ Further, molecular simulations reveal up to residue Cp149 could reach the pore.⁸² This result along with our observations of limited SBA-GFP binding to pre-assembled Cp149 and Cp142 VLPs is intriguing. A possible explanation may be that a portion of the linker domain (in a capsid context) interacts with either base or capping chain residues that form the CAM pocket, ultimately blocking critical residues required for coordinating a CAM. In a dimer context, these interactions are non-existent enabling co-assembly with SBA-GFP and decoration of the VLP.

### EXAMPLES

### Example 1: Preparation of Tripartite Constructs With an SBA CAM

SBA-cargo are produced by attaching an SBA molecule with linker followed by conjugation of a GFP.

### SBA Synthesis and Characterization

### Synthesis of SPDP-PEG4-SBA

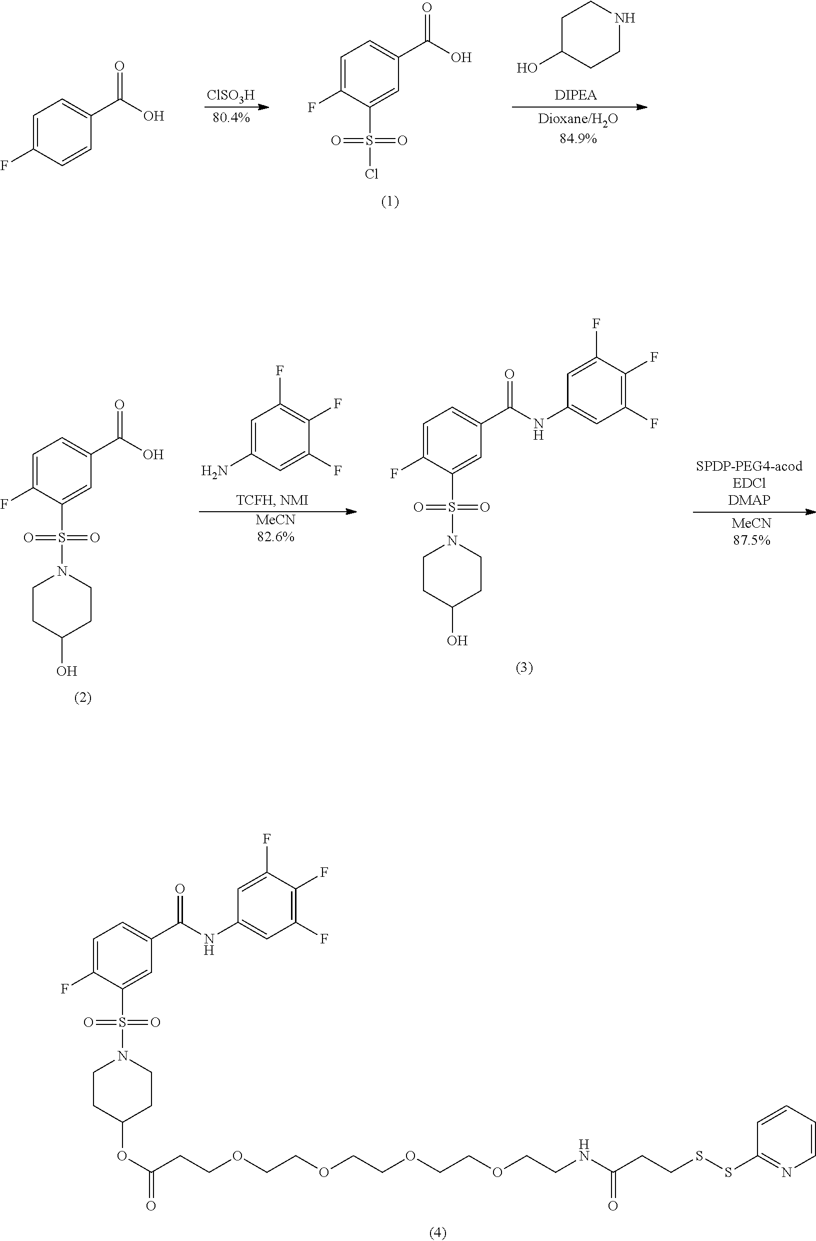

Sulfamoylbenzamide (SBA) NVR 3-778 was prepared via the previously published route albeit with modified reaction conditions¹. The chlorosulfonylation reaction under refluxing chlorosulfonic acid provided sulfonyl chloride (1), followed by the addition of 1,4-piperidinol and N,N-diisopropylethylamine to afford the sulfonamide intermediate (2). The amide coupling reaction was performed using coupling agent chloro-N,N,N′,N′-tetramethylformamidinium hexafluorophosphate (TCFH) with N-methyl imidazole (NMI) to form NVR 3-778 (3)². Steglich esterification with SPDP-PEG4-acid is used to install the linker at the piperidinol alcohol, which provides the final product, SPDP-PEG4-SBA (4).

### Synthesis of Maleimide-PEG4-SBA

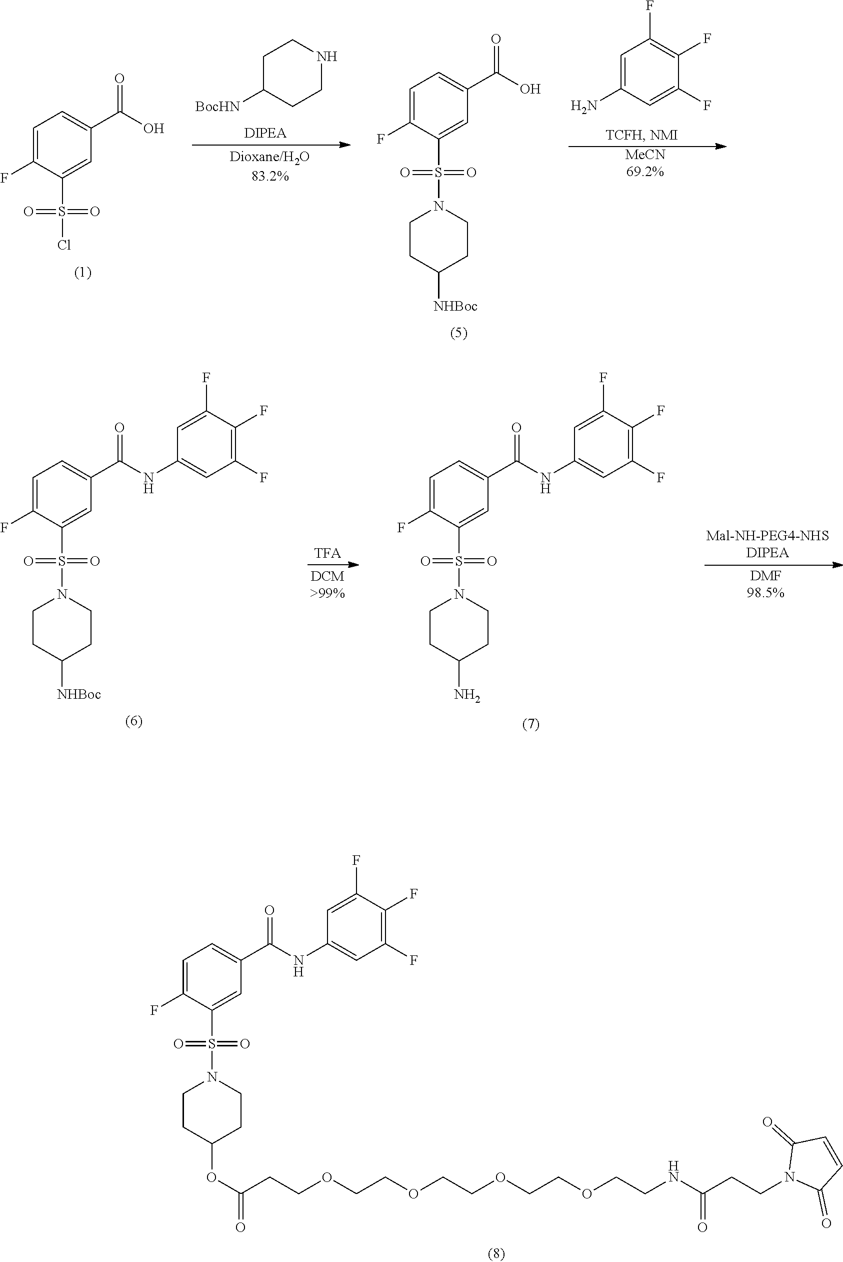

Sulfonyl chloride (1) was synthesized as described above. Sulfonamide (5) was synthesized by the addition of 4-Boc-aminopiperidine and N,N-diisopropylethylamine, followed by an amide coupling reaction using chloro-N,N,N′,N′-tetramethylformamidinium hexafluorophosphate (TCFH) with N-methyl imidazole (NMI) provided SBA derivative (6). Deprotection of the Boc group in trifluoroacetic acid and dichloromethane affords free amine (7), which was directly submitted to an amide coupling with Mal-NH-PEG4-NHS ester to afford the final product Mal-PEG4-SBA (8).

### Attachment of the CAM Tag to a Protein Cargo

For these experiments, GFP was used as an exemplary cargo. The GFP used in this study has a single accessible cysteine. A variant of sfGFP (PDB: 2B3P) (Pédelacq, J.-D., et al., Engineering and characterization of a superfolder green fluorescent protein. Nat. Biotechnol. 2006, 24 (1), 79-88).

was engineered with flexible linkers on its N- and C-terminus containing an accessible cysteine. Only the N-terminal cysteine is active. The engineered sfGFP was transformed in an E. coli overexpression system. 250 mL Luria Broth cultures containing 100 μg/mL carbenicillin were grown at 37□C till reaching an optical density (OD600) of ˜0.8. Expression was induced by addition of 1 mM IPTG and grown overnight. The GFP was purified by harvesting and resuspending the cells in lysis buffer containing 50 mM HEPES-pH 7.5, 150 mM NaCl, 5 mM DTT, 0.01 mg/mL DNase, 0.10 mg/mL RNase). Cells were lysed by emulsification and clarified using two rounds of centrifugation. Clarified supernatant was further purified by nickel affinity and by size-exclusion chromatography using a handpacked S300 10/300 column. The quality of the final product was confirmed by LC-MS and SDS-PAGE. Protein concentrations were calculated using extinction coefficients 27,529 M−1 cm−1 (ε280) and 61,941 M−1 cm−1 (ε488).

For conjugation, GFP was incubated with two-fold molar excess of SBAₗᵢₙₖₑᵣ in 50 mM HEPES, pH 7.5 at 4° C. overnight. Excess ligand is purified away using a PD10 column and the final product is confirmed by liquid chromatography-mass spectrometry (LC-MS) and size exclusion chromatography (SEC). Mass spectrometry shows a single species predicted at 28,622 Da (net gain of 767 Da compared to unmodified GFP), equivalent to one SBA ligand per GFP. Purified SBA_{SPDP}-GFP ligand elutes as a multiple peaks on a Superose 6 10/300 GL column ranging from 16-20 mL. The main peak, corresponding to monomeric GFP, elutes at 18 mL.

Multiple SBA conjugates were synthesized. Conjugates were purified and evaluated for activity to co-assemble with HBV Cp and to bind to preassembled HBV VLPs. Using size exclusion chromatography (SEC), we have shown a concentration dependence to binding. Most cargo molecules are packaged inside the VLP with few displayed on its exterior. External ligands can be depleted and purified from the cargo-loaded VLP under reducing conditions. We have confirmed packaging of cargo proteins by SDS-PAGE, western blot, resistive pulse sensing. The SBA conjugates have no effect on VLP quaternary structure as seen by electron microscopy.

As described in more detail in the following examples, SBA_{SPDP}-GFP co-assembles with dimeric assembly domain, Cp149, and retains its ability to speed up assembly of Cp. Assembly kinetics are dependent on the concentration of SBA_{SPDP}-GFP added to the reaction mixture. Higher stoichiometries of ligand were inversely proportional to rate and completion of VLP assembly.

Evaluation of co-assembly products by SEC show a concentration dependence to SBA_{SPDP}-GFP binding. Higher stoichiometries of inputed SBA_{SPDP}-GFP into the co-assembly reaction led to higher loading densities of cargo molecules to the VLP.

External decoration of pre-assembled particles: Binding of SBA_{SPDP}-GFP to purified preassembled VLPs showed an upper limit to external decorations of cargo molecules. Regardless of the amount of SBA_{SPDP}-GFP added to the reaction mixture, 7-10 cargo proteins were associated. External GFP decorations can be removed with treatment of a reducing agent, beta-mercaptoethanol (BME).

### Example 2: Comparison of Tripartite Constructs Using HAP and SBA

Tripartite constructs were prepared with different CAM portions. Tripartite constructs having HAP as the CAM unit (HAP-cargo) are produced by attaching an HAP molecule with linker followed by conjugation of a GFP following the procedure in Example 1.

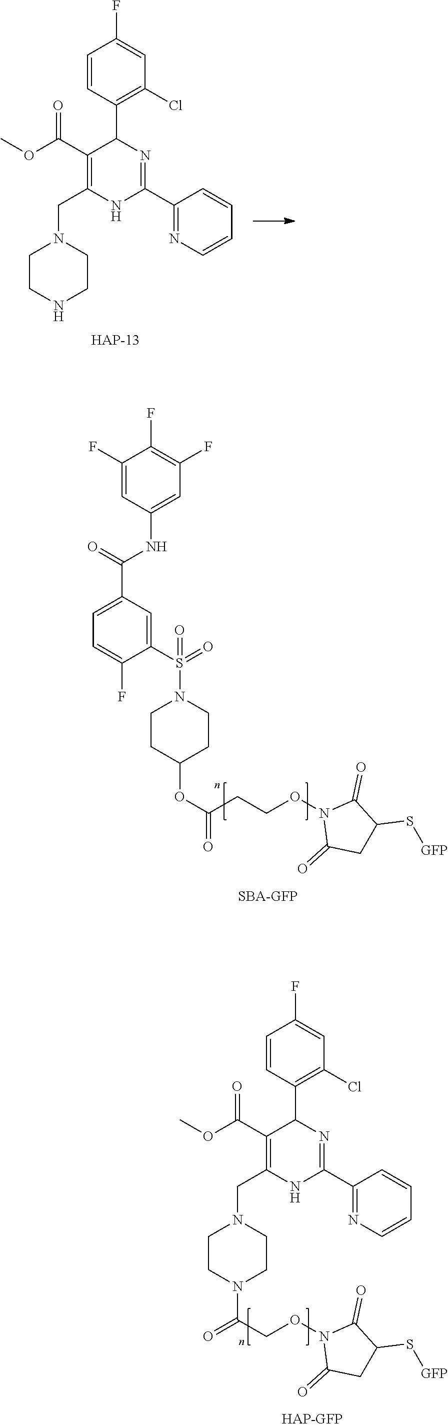

The performance of the tripartite constructs with an SAB as the CAM (SAB-cargo) of Example 1 and the HAP construct to package protein cargo (co-assembly with capsid proteins) and externally decorate (binding to pre-assembled capsid) HBV VLPs is compared.

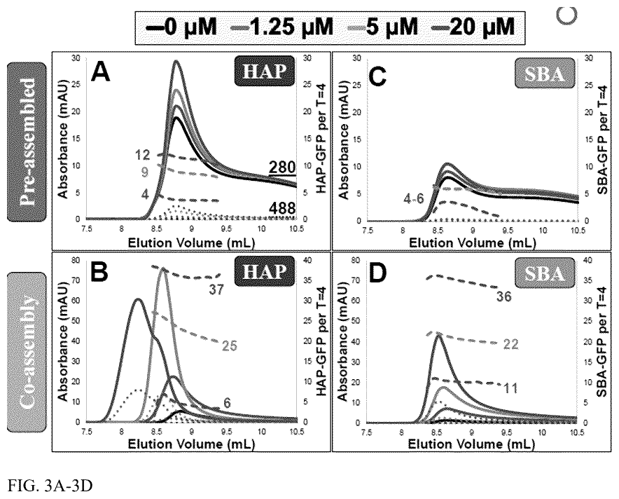

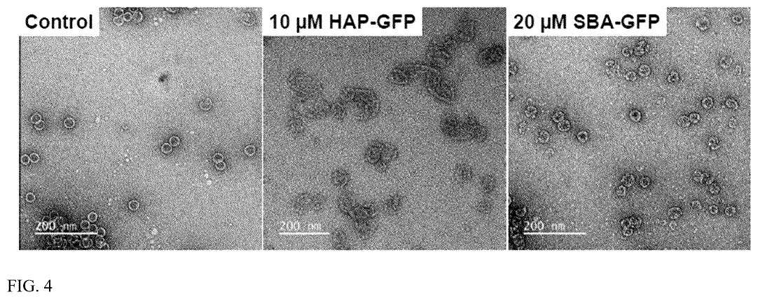

To evaluate the tripartite constructs performance in externally decorating HBV VLPs, HAP-tagged GFP and SBA-tagged GFP were added to crosslinked pre-assembled Cp150 capsids. FIGS. 3A and 3C show size exclusion chromatography (capsid peak shown) of HAP—(FIG. 3A) or SBA-GFP (FIG. 3C). HAP-GFP binding to Cp150 capsid was dose-dependent, while SBA-GFP had limited activity. VLPs were decorated with up to 12 cargo proteins for HAP-tagged cargo. SBA-tagged cargo were limited to half that amount, regardless of concentration. (FIG. 3A and 3C)

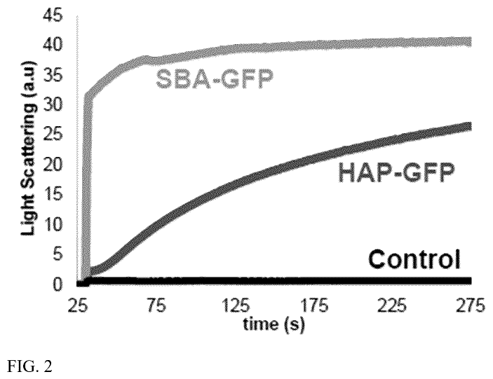

In co-assembly reactions, HBV Cp149 dimers were mixed with 10 μM CAM-cargos SAB-GFP of Example 1 and the HAB-GFP and were monitored by right-angle light scattering. CAM-cargos increase rate and extent of VLP assembly. (FIG. 2). In co-assembly, both CAMs had similar amounts of association. The results are shown in FIG. 3B and 3D. However, HAP-tagged cargos enriched for aberrant structures. SBA-tagged cargo in co-assembly reaction were likely to be packaged internally and externally.

FIG. 4 depicts transmission electron microscopy images of CAM-GFP-VLP complexes. Co-assembly with HAPs misassembled Cp and favored aberrant structures, while SBAs maintained spherical VLPs. HAP-GFP drove assembly of Cp aggregates while SBA-GFP maintained VLP shape.

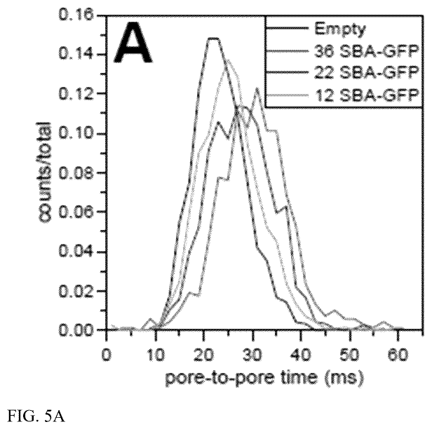

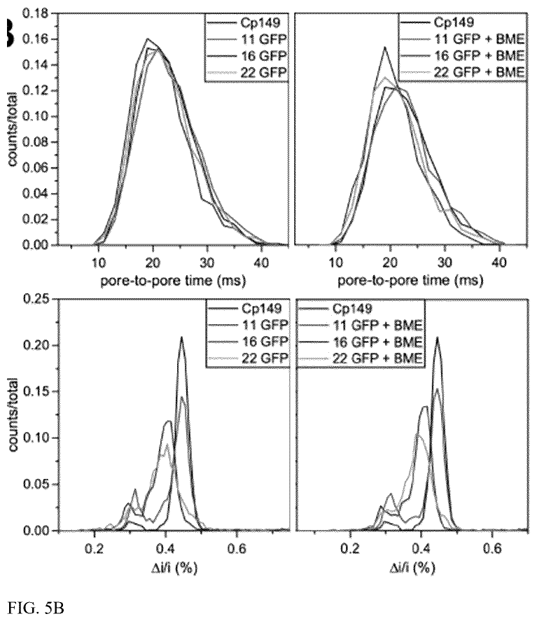

To evaluate whether during co-assembly the SBA-GFPs are located inside or outside the VLP, resistive pulse sensing of SBA-GFP-VLPs was conducted. (FIGS. 5A-B) Pore-to-pore (P2P) time is dependent on particle surface charge, while Δi/i is proportional to particle volume. As shown in FIG.5A, increasing amounts of associated SBA-GFP increase VLP surface charge, suggesting external decorations. P2P times of complexes were similar regardless of treatment with and without reducing agent BME (FIG. 5B, top). No change was observed in the Δi/i, though intermediate material was observed (FIG. 5B, bottom).

### Example 3—Synthesis of SBA-SPDP-GFP

A representative CAM-E, NVR 3-778 (also known as SBA_R01) was synthesized and was covalently connected to a 26 Å pegylated succinimidyl 3-(2-pyridyldithio)propionate (SPDP) bifunctional crosslinker. The parent SBA_R-01 molecule contains a piperidine with a hydroxyl group at the 4th-position which was functionalize via Steglich esterification with the carboxylic acid end of the SPDP linker. The opposing end of the SPDP linker provided a pyridyldithiol group enabling reversible conjugation to protein cargo, a superfolder GFP (sfGFP) carrying an accessible cysteine residue. sfGFP was attached to SBA-SPDP through disulfide exchange. The final product SBA-SPDP-GFP (hereafter referred to as SBA-GFP) was confirmed by LC-MS and no detectable unlabeled sfGFP.

The scheme is shown below:

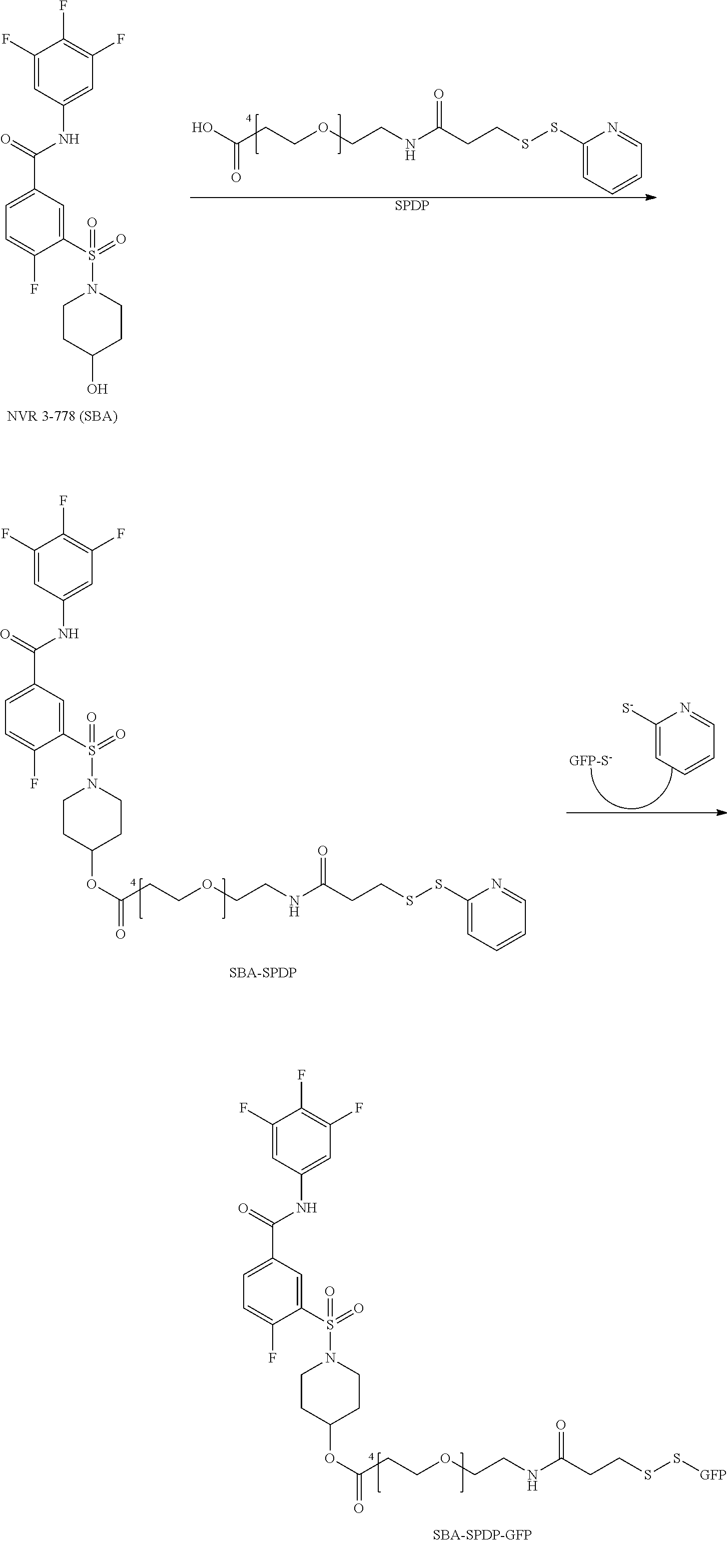

### Example 4: Evaluation of Linker and Cargo Additions to CAM on Binding and Assembly

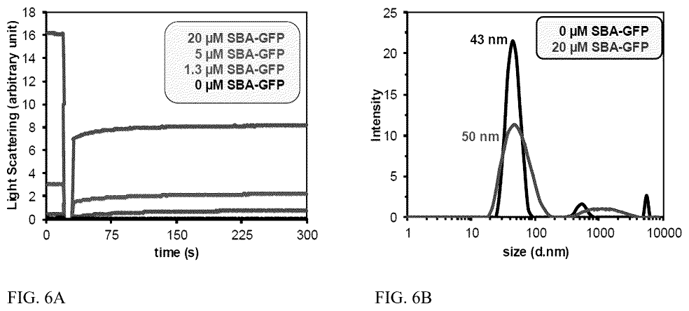

To ensure the covalent additions of a linker and sfGFP to the SBA had any effect on binding to Cp and assembly of empty VLPs, they were assessed by right-angle light scattering. Dimeric Cp149 was mixed with varying amounts of SBA-GFP and assembly was monitored in real-time by increasing the solutions ionic strength to 300 mM NaCl. Compared to Cp-only controls, SBA-GFP driven assembly of empty VLPs was concentration dependent leading to a higher titer of assembled VLP. Assembly was VLPs in the presence of SBA-GFP achieved near completion within a few minutes. The amount of assembled VLP assembled was SBA-GFP concentration dependent. FIG. 6A. While VLPs assembled with SBA-GFP roughly maintained their size, the VLPs co-assembled with 20 μM SBA-GFP (magenta) exhibited a broader range of sizes compared the capsid control (black), with the median at 50 and 43 nm, respectively. (FIG. 6B) and were similar in shape to the capsid control (FIG. 7).

In co-assembly experiments, mixtures of 5 μM Cp149 and increasing amounts SBA-GFP was assembled by raising the ionic strength of the solution to 300 mM NaCl. Assembly products were evaluated by size exclusion chromatography. Using a superose 6 increase column, VLPs maximally elute at 8.6 mL and are baseline resolved from free Cp149 dimer and SBA-GFP which roughly elute at 18 mL. By monitoring elution of total protein and SBA-GFP by using absorbance at 280 nm and 488 nm, respectively, enabled us to track SBA-GFP binding and calculate the number of bound SBA-GFPs per VLP after correcting the absorbance at 280 nm for GFP contributions as previously described.

### Example 5: Evaluation of Concentration Dependence During Co-Assembly

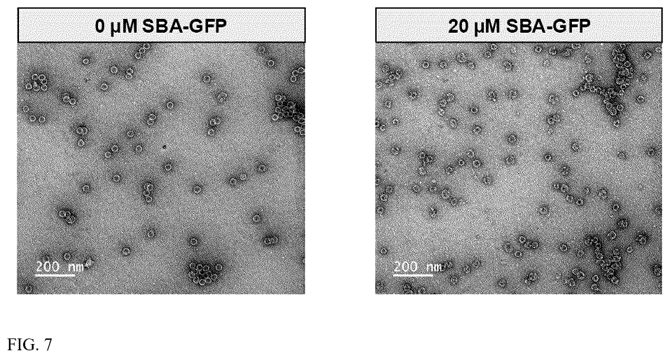

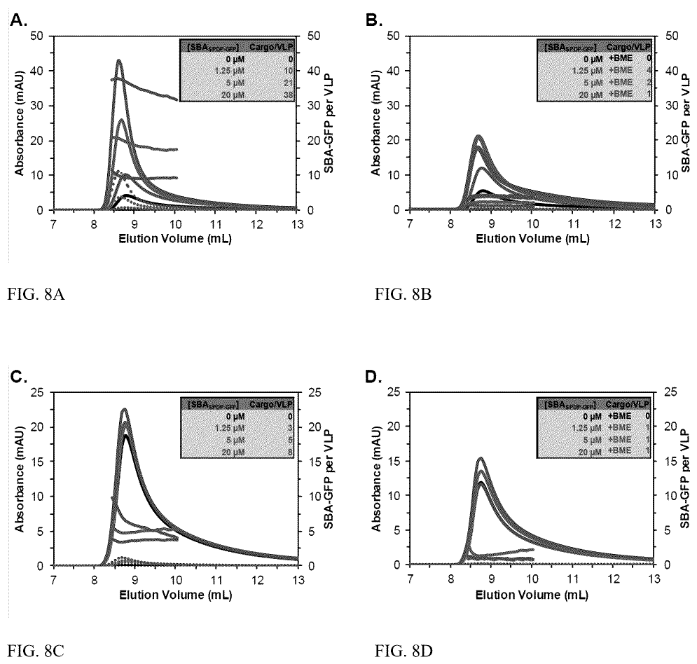

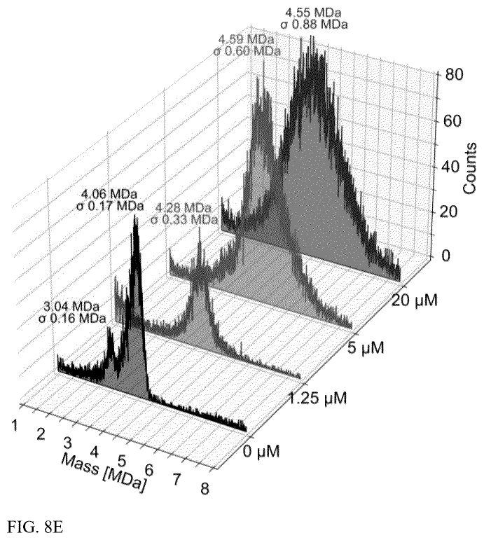

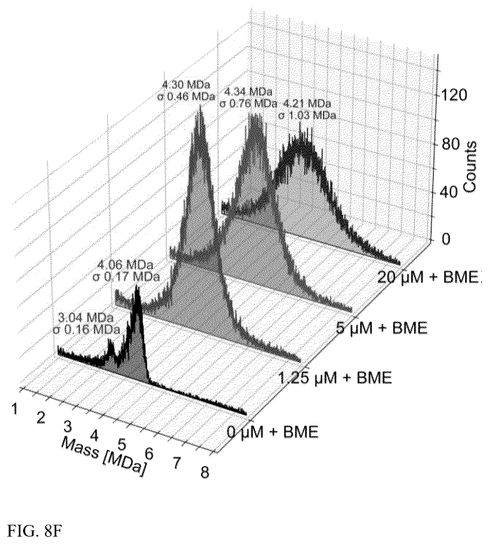

Co-assembly mixtures of Cp149 and varying amounts of SBA-GFP showed a strong positive relationship between input SBA-GFP and amount of assembled VLP (at the expense of free dimer). Additionally, a concentration dependence to binding was observed by the co-elution of SBA-GFP with the VLP peak (FIG. 8A). Treatment of co-assembled VLPs with BME had complete loss of 488 nm signal eluting with the capsid peak, suggesting that practically all SBA-GFPs were preferentially displayed on the exterior of the VLP (FIG. 8B). Applicant speculated fast binding kinetics of SBA-GFP may not provide enough time for the VLP to assemble around an associated SBA-GFP. Interestingly, changing NaCl or temperature assembly conditions had practically no effect the amount of packaged SBA-GFP.

To confirm the SBA-GFP per VLP ratios, the mass distributions of coassembled VLPs with SBA-GFP before (FIG. 8E) a and after BME treatment (FIG. 8F) was monitored using mass photometry. In mass photometry, the mass of individual particles is measured by the interference of light scattered by a particle on a surface and light the reflected by that surface.77-78 The VLP control clearly defined T=3 and T=4 capsid morphs with a mass of approximately 3 and 4 MDa, respectively. Co-assembly with increasing amounts of SBA-GFP typically lead to higher mean mass in the gaussian distribution, similar to our absorbance measurements. At a 4:1 (1.25 μM) and 1:1 (5 μM) dimer to SBA-GFP ratio, we observed a maximum of 10 and 21 SBA-GFPs per VLP measured by absorbance. Similarly, under these same conditions mass photometry predicted a mean mass of 7 and 19 SBA-GFPs. Though we were unable to resolve T=3 and T=4 VLPs, due to the broadening of the VLP peaks. Broadening of the VLP population with SBA-GFP binding was similarly observed with HAP-GFPs resolved by charge detection mass spectrometry. Mass predictions by photometry at 20 μM (1:4 ratio) were much lower than what was calculated by absorbance. Additionally, after BME treatment, 5-10 GFPs were estimated to be associated with the VLPs, a slight overestimation to our absorbance measurements.

### Example 6: Evaluation of Concentration Dependence in Binding to Pre-Assembled Cp149 VLPs

Cp149 VLPs were assembled and purified, then mixed with varying concentrations of SBA-GFP. Interestingly, SBA-GFPs did not bind well even with increasing the initial input concentration several fold, with very few SBA-GFPs associating to the VLP at each tested concentration (FIG. 8C). As expected, treating these samples with BME removed all SBA-GFPs from the exterior of the pre-assembled VLP (FIG. 8D). These results were counterintuitive, while co-assembly decorated the VLPs with high amounts of SBA-GFP, very few bind to intact VLPs—a paradox that may suggest intrinsic structural or steric constraints in the context of an intact VLP.

### Example 10—Analysis of Capsid Protein Constructs—Cp149 and Cp142

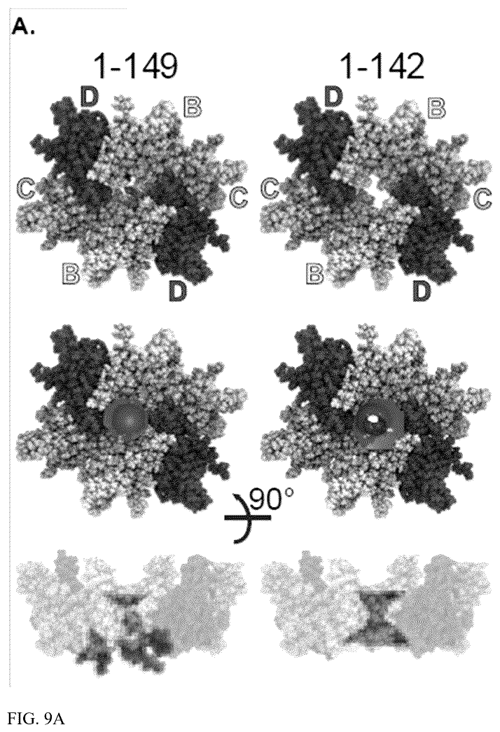

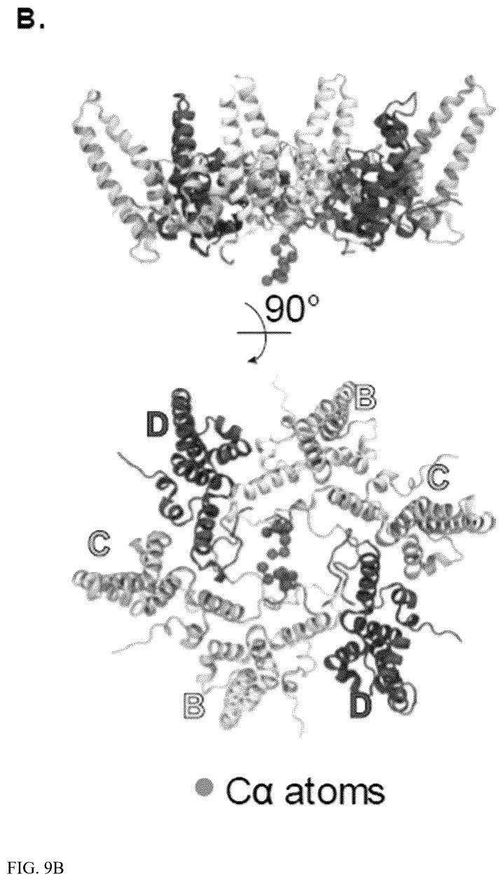

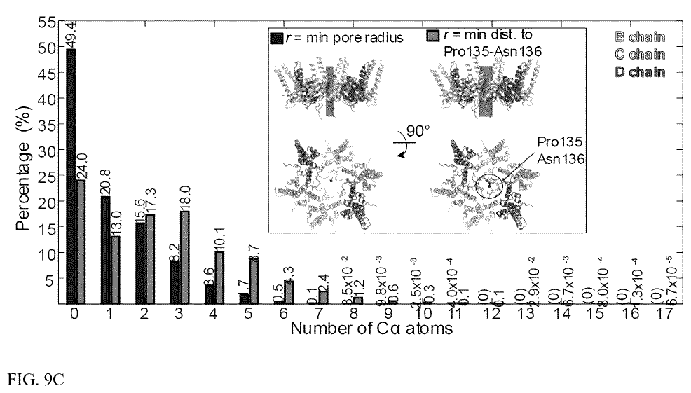

We were intrigued by our observations of Cp149 coassemblies with SBA-GFPs. Solution conditions or temperature had no effect on packaging efficiency. We hypothesized, making more space in the VLP may provide enough room for SBA-GFPs to package inside the VLP, but also improve decorating the capsid exterior. This hypothesis was also based on previous studies showing the linker domain on the C-terminus of full-length HBV Cp183, that primarily resides on the inside of the VLP as a disordered region, can extend and protrude outside the VLP. Additionally, the linker region is within the proximity of the SBA CAM binding site. We used molecular dynamics to calculate the radius of the quasi-sixfold pores determined by HOLE (FIG. 9A). With full length assembly domain, Cp149, complete occlusion of the pore was observed by C-terminal linker residues. Our studies also showed this region can extend all beyond the 149^{th} residue, up to 17 residues in some cases (FIG. 9B). We removed residues 143-149 for our molecular dynamic simulations and we observed the truncation enabled HOLE to detect pore opening, suggesting that linker residues crowded the pore exit (FIG. 9A). Finally, to determine how many linker residues were predicted to be in the path of the pore (FIG. 9C), we estimated the pore radius based on the minimum pore distance and the minimum distance between residues Pro135 and Asn136. The former provided an underestimation while the latter gave an overestimation of the pore radius, respectively. The two estimations, due to the asymmetric nature of the quasi six-folds, showed the dynamic nature of linker conformers to block the pore exit way. These observations are in agreement with prior simulations confirming the capsid flexibility for function.

Dimeric Cp149 (SEQ ID NO:3) was expressed and purified using standard practices as previously described. (Hussain, T, et al., Chemically Tagging Cargo for Specific Packaging inside and on the Surface of Virus-like Particles. ACS Nano 2024).

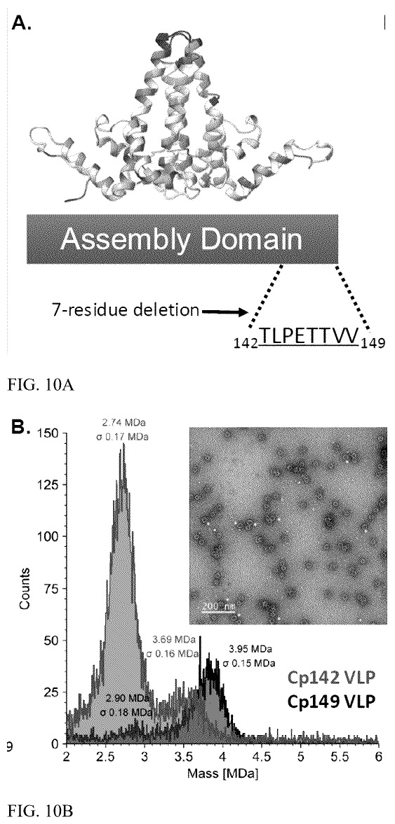

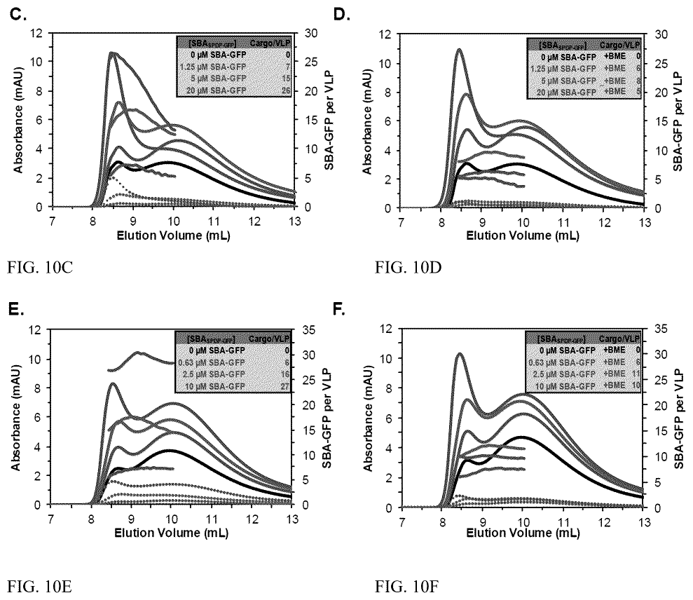

To evaluate the impact of the Cp in combination with the tripartite tool, the C-terminal residues of Cp149 (143-149) were deleted from the pET11c vector expressing Cp149 using the In-Fusion seamless cloning system (Takara). FIG. 10A. The Cp142 gene (SEQ ID NO: 6) was confirmed by Sanger Sequencing (Eurofins Genomics) using T7 promoter and T7 terminator primers. The new plasmid was transformed into BL21 DE3 competent cells, overexpressed, and purified using the same standard procedures as Cp149.

The primers used for the deletion were:

sense:
(SEQ ID NO: 7)
5′-CCCTATCTAATCGACACTTCCGGAGACTACG-3′
antisense:
(SEQ ID. NO: 8)
5′-GTCGATTAGATAGGGGCATTTGGTGGTC-3′

Concentration of capsid proteins were determined using an extinction coefficient (□280) of 66,302 M−1 cm−1. Dimers were stored at 4□C until use for in vitro assays.

Assembly of VLPs. Cp149 and Cp142 dimers were buffer exchanged from storage solution to 50 mM HEPES-pH 7.5 using a PD10 column. To assemble VLPs, dimers were assembled by mixing 40 μM dimer with an equivalent volume of assembly buffer (50 mM HEPES-pH 7.5, 600 mM NaCl). Final assembly conditions were 20 μM dimer, 50 mM HEPES-pH 7.5, and 300 mM NaCl). Assembly reactions were allowed to incubate 24-48 hours at RT before being purified from unassembled dimer by size exclusion using a handpacked S300 10/300 column. VLPs were stored at 4° C. until use for in vitro assays.

Unlike Cp149, which assembles into 95% T=4 VLPs, Cp142 dimers assembles into roughly 85% T=3 VLPs (FIG. 10B). This is consistent with prior work showing the length of the linker domain acts as a morphogenic switch.⁵³

First, the capacity of SBA-GFP to bind to pre-assembled Cp142 VLPs was evaluated. To our surprise, we observed SBA-GFP had comparable binding to pre-assembled Cp149 VLPs. In our chromatographs, the T=3 VLPs elute at approximately 10.5 mL, shortly after T=4 VLPs. The peak corresponding to T=3 Cp142 VLPs outweighed the T=4 peak, though could be resolved allowing us to calculate SBA-GFP per VLP.

Next, SBA-GFP co-assembly with Cp142 dimers was evaluated and observed a similar concentration dependent to binding. Though generally less binding at each concentration tested was seen compared with co-assembly with Cp149 dimers (FIG. 10C and 10E). Additionally, as we increased the SBA-GFP concentration in reaction mixtures, preference for assembly of capsids with T=4 geometry was apparent. We speculate that to accommodate packaged GFP, the capsid with more space was preferred.

Treating reactions with BME reduced the number of bound SBA-GFP to the VLP, though modest signal was detected (FIG. 10D and 10F). On average a humble six SBA-GFPs were packaged. This was 6× more than what we observed with Cp149 co-assembly.

We next theorized that packaging SBA-GFP clusters could have synergistic effects with Cp142 dimers and ultimately would improve efficiency. To this end, we preformed SBA-GFP complexes and fed them into a co-assembly reaction with Cp142 dimers. At each concentration tested, we associated less SBA-GFP to the Cp142 VLP compared to Cp149 co-assemblies with SBA-GFP clusters. To our surprise, we treated Cp142 VLPs with BME and found that only 10 SBA-GFPs packaged at a concentration of 20 μM. This suggests the space of the VLP has no effect on packaging SBA-GFP clusters.

### Example 11: Materials and Methods

In binding experiments, purified VLPs were diluted to 5 μM and mixed with the desired final concentration of SBA-GFP. Solutions conditions were typically in 50 mM HEPES-pH 7.5 and 300 mM NaCl unless otherwise specified. In co-assembly experiments, 10 μM Cp149 or Cp142 dimers were mixed 2× the desired concentration of SBA-GFP. Co-assembly was initiated by mixing an equivalent volume of 50 mM HEPES-pH 7.5-600 mM NaCl. Final conditions were 5 μM dimer, 50 mM HEPES-pH 7.5, 300 mM NaCl and varying SBA-GFP. Assemblies were incubated overnight before purification or characterization. Solution conditions that are varied in terms of NaCl concentration or temperature is specified in text.

The number of SBA-GFP per VLP in the binding and co-assembly reactions were evaluated by size exclusion chromatography. After an overnight incubation, the samples were loaded onto a factory-packed Superose 6 increase 10/300 column (Cytiva) plumbed onto a Shimadzu HPLC system equipped with multiwavelength absorbance detector. The mobile phase was consistent with the final HEPES and NaCl conditions (50 mM HEPES, pH 7.5-300 mM NaCl). VLPs elute maximally at 8.6 mL, whereas unassembled dimer and free SBA-GFP elute at approximately 17 mL. To calculate the number of SBA-GFP per VLP, the contribution of GFP to the A280 was subtracted, then the molar ratio of SBA-GFP to VLP was calculated.70

To determine the number of packaged SBA-GFP, following overnight incubation, assembly reactions were treated with 10 mM BME and incubated for an additional 16-24 hours. Treated samples were evaluated by size exclusion as described above.

Light Scattering Experiments: Assembly of dimeric Cp149 and SBA-GFP was monitored by 90□ light scattering using a photon technology international fluorometer. Excitation and emission monochromators were set to 320 nm to minimize SBA-GFP absorbance contribution. 10 μM purified Cp149 was mixed with increasing amounts of SBA-GFP (or unmodified SBA, NVR3-778) for approximately 20 seconds before adding an equivalent volume of 50 mM HEPES-pH 7.5, 600 mM NaCl to initiate assembly (monitored for 5 minutes). Final conditions are 5 μM Cp149, 50 mM HEPES-pH 7.5, 300 mM NaCl, and the indicated amount of SBA-GFP. Each trace was independently repeated three times. A representative trace is shown. Dynamic light scattering measurements were performed on a Malvern Zetasizer (Physical Biochemistry Instrumentation Facility, Indiana University). Samples were assembled as described in the binding experiments method section. The purified VLPs (without and with SBA-GFP associated) were evaluated for average size and polydispersity.

### Transmission Electron Microscopy.

Samples were assembled and purified as previously described and examined by negative stain electron microscopy. 4 μL of purified VLPs were applied to glow discharged copper grids (Electron Microscopy Sciences), washed with 4 μL of water, and stained with 4 μL of 0.75% uranyl formate prior and allowed to air-dry for 24 hours prior to examination. Micrographs were taken at 30 k magnification using a JEOL JEM 1400plus 120 kV TEM equipped with a Gatan One View CMOS (Electron Microscopy Center, Indiana University).

### Mass Photometry.

The mass distribution of coassembled VLPs were quantified using a REFEYN TwoMP mass photometer (working range of 30 kDa-5000 kDa). Measurements were typically recorded for 60 seconds and using buffer-free find focus. HBV Cp149 assembles into two distinct capsids, a roughly 3 MDa T=3 and 4 MDa T=4 particle, where the latter is the predominant population (˜95%). This enables an approximation of the number of SBA-GFPs (˜28 kDa) bound to a VLP. 4 μM of purified VLP was typical for these measurements. To measure the growing mass of SBA-GFP complexes, 0.4 mM NiCl2 was mixed with 40 μM SBA-GFP and incubated at 4□C. An aliquot was taken at the indicated time and diluted to 1 or 4 μM for the measurement. Higher concentrations of sample allowed gaussian fitting for higher mass complexes not observed with 1 μM.

Table of Sequences

| SEQ | | |
|---|---|---|
| ID | | |
| NO | Name | |
| 1 | HBV | 1 mdidpykefg atvellsflp sdffpsvrdl ldtaaalyrd alespehcsp hhtalrqail |
| | Cp183 | 61 cwgdlmtlat wvgtnledpa srdlvvsyvn tnvglkfrql lwfhiscltf gretvleylv |
| | AA | 121 sfgvwirtpp ayrppnapil stlpettvvr rrgrsprrrt psprrrrsqs prrrrsqsre |
| | sequence | 181 sqc |
| 2 | HBV | atggacattgacccttataaagaatttggagctactgtggagttactctcgtttttgccttctgacttc |
| | Cp183 | tttccttccgtacgagatcttcttgataccgccgcagctctgtatcgggatgcattagagtctcctgag |
| | gene | cactgcagccctcaccatactgccttaaggcaagcaattctttgctggggagacttaatgactctagct |
| | | acctgggtgggtactaatttagaagatccagcatctagggacctagtagtcagttatgtcaacactaat |
| | | gtgggcctaaagttcagacaattattgtggtttcacatttcttgtctcacttttggaagagaaacggtt |
| | | ctagagtatttggtgtcttttggagtgtggattcgcactcctccagcttatagaccaccaaatgcccct |
| | | atcctgtcgacgcttccggagactactgttgttcgtcgccgtggccgttccccgcgtcgccgtactccg |
| | | tcgccgcgtcgccgtcgctctcaatcgccgegtcgccgtcgttctcaatctcgtgaatctcaatgt |
| 3 | HBV | 1 mdidpykefg atvellsflp sdffpsvrdl ldtaaalyrd alespehcsp hhtalrqail |
| | Cp149 | 61 cwgdlmtlat wvgtnledpa srdlvvsyvn tnvglkfrql lwfhiscltf gretvleylv |
| | AA | 121 sfgvwirtpp ayrppnapil stlpettvv |
| | sequence | |
| 4 | HBV | atggacattgacccttataaagaatttggagctactgtggagttactctcgtttttgccttctgacttc |
| | Cp149 nt | tttccttccgtacgagatcttcttgataccgccgcagctctgtatcgggatgcattagagtctcctgag |
| | sequence | cactgcagccctcaccatactgccttaaggcaagcaattctttgctggggagacttaatgactctagct |
| | | acctgggtgggtactaatttagaagatccagcatctagggacctagtagtcagttatgtcaacactaat |
| | | gtgggcctaaagttcagacaattattgtggtttcacatttcttgtctcacttttggaagagaaacggtt |
| | | ctagagtatttggtgtcttttggagtgtggattcgcactcctccagcttatagaccaccaaatgcccct |
| | | atcctgtcgacgcttccggagactactgttgtt |
| 5 | HBV | 1 mdidpykefg atvellsflp sdffpsvrdl ldtaaalyrd alespehcsp hhtalrqail |
| | Cp142 | 61 cwgdlmtlat wvgtnledpa srdlvvsyvn tnvglkfrql lwfhiscltf gretvleylv |
| | AA | 121 sfgvwirtpp ayrppnapil st |
| | sequence | |
| 6 | HBV | ATGGACATTGACCCTTATAAAGAATTTGGAGCTACTGTGGAGTTACTCTCGTTTT |
| | Cp142 nt | GGGGAGACTTAATGACTCTAGCTACCTGGGTGGGTACTAATTTAGAAGATCCAG |
| | sequence | GAGTGTGGATTCGCACTCCTCCAGCTTATAGACCACCAAATGCCCCTATCCTGTC |
| 7 | Sense | 5′-CCCTATCTAATCGACACTTCCGGAGACTACG-3′ |
| | Primer | |
| 8 | Antisens | 5′-GTCGATTAGATAGGGGCATTTGGTGGTC-3′ |
| | e Primer | |

### REFERENCES

Lee, Y. H.; Cha, H. M.; Hwang, J. Y.; Park, S. Y.; Vishakantegowda, A. G.; Imran, A.; Lee, J. Y.; Yi, Y. S.; Jun, S.; Kim, G. H.; Kang, H. J.; Chung, S. J.; Kim, M.; Kim, H.; Han, S. B. Sulfamoylbenzamide-Based Capsid Assembly Modulators for Selective Inhibition of Hepatitis B Viral Replication. ACS Med Chem Lett 2021, 12 (2), 242-248. https://doi.org/10.1021/acsmedchemlett.0c00606.
Beutner, G. L.; Young, I. S.; Davies, M. L.; Hickey, M. R.; Park, H.; Stevens, J. M.; Ye, Q. TCFH-NMI: Direct Access to N-Acyl Imidazoliums for Challenging Amide Bond Formations. Org Lett 2018, 20 (14), 4218-4222. https://doi.org/10.1021/acs.orglett.8b01591.

I claim:

1. A tripartite tool for attaching a protein cargo of interest to the inside and outside of an HBV virus-like particle (VLP), composed of a capsid assembly modulator (CAM), a linker, and a protein of interest, wherein the CAM is attached to the linker and the protein of interest is attached at the opposite end of the linker.

2. The tripartite tool of claim 1 wherein the CAM comprises sulfamoylbenzamide (SBA).

3. The tripartite tool of claim 1 wherein the length of the linker extends from the capsid wall to the capsid lumen.

4. The tripartite tool of claim 4 wherein the linker is terminated with a maleimide.

5. The tripartite tool of claim 2 wherein the SBA is chemically attached to the linker.

6. The tripartite tool of claim 1 wherein the protein of interest contains a single reactive cysteine.

7. The tripartite tool of claim 6 wherein the protein if interest is attached to linker of claim 4 through a reaction of the maleimide of the linker and the reactive cystine of the protein of interest.

8. A method of attaching a protein cargo to the exterior of an HBV virus-like particle (VLP), comprising the steps of
preparing the tripartite tool of claim 1, and
mixing the tripartite tool with pre-assembled HBV VLP capsids.

9. A method of attaching a protein cargo of interest to the interior of an HBV virus-like particle (VLP), comprising the steps of
preparing the tripartite tool of claim 1,
mixing the tripartite tool with HBV capsid protein (CP) homodimer, and
inducing assembly of the HBV VLP capsids.

10. The method of claim 9 further comprising the step of removing any protein of interest present on the exterior of the HBV VLP capsids by treating the assembled HBV VLP capsids with a reducing agent.

11. The tripartite tool of claim 1, wherein the CAM is a sulfamoylbenzamide (SBA) of formula (I) 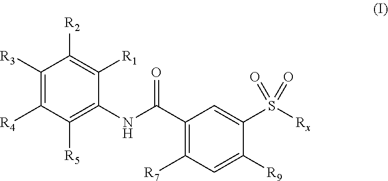
including hydrates, solvates, pharmaceutically acceptable salts, prodrugs and complexes thereof, wherein:
Rₓ is a formula of (I-1), (I-2), or (I-3) 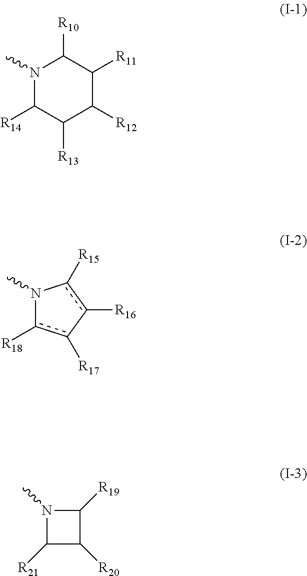
the wave-line denoting the connection to Formula I;
wherein
R₁ is hydrogen;
R₂ is selected from a group consisting of hydrogen, methyl, trifluoromethyl, fluorine, and chlorine;
R₃ is selected from a group consisting of hydrogen, methyl, fluorine, and chlorine;
R₄ is selected from a group consisting of hydrogen, fluorine, chlorine, and methyl;
R₅ is selected from a group consisting of hydrogen and chlorine;
R₇ is selected from a group consisting of hydrogen, chlorine, fluorine, and bromine;
R₉ is selected from a group consisting of hydrogen, methyl, fluorine, and chlorine; and
wherein when Rx is a formula of (I-1), one of R₁₀, R₁₁, R₁₂, R₁₃, and R₁₄ is OH and the remaining of R₁₀, R₁₁, R₁₂, R₁₃, and R₁₄ are each hydrogen;
when Rx is a formula of (I-2), one of R₁₅, R₁₆, R₁₇, and R₁₈ is OH, and the remaining of R₁₅, R₁₆, R₁₇, and R₁₈ are each hydrogen;
when Rx is a formula of (I-3), one of R₁₉, R₂₀, and R₂₁ is OH, and the remaining of R₁₉, R₂₀, and R₂₁ are each hydrogen; [symbol]
markings represent single or double bonds.

12. The tripartite tool of claim 11, wherein R₂, R₃, and R₄ are each fluorine.

13. The tripartite tool of claim 11, wherein R₁ and R₅ are each hydrogen.

14. The tripartite tool of claim 11, wherein R₇ is hydrogen and R₉ is fluorine.

15. The tripartite tool of claim 14, wherein Rₓ is

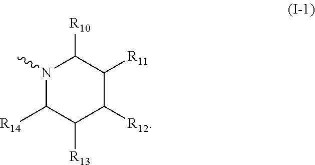

16. The tripartite tool of claim 15, wherein R₁₀, R₁₁, R₁₃, and R₁₄ are each hydrogen and R₁₂ is OH.

17. The tripartite tool of claim 11, wherein Rₓ is

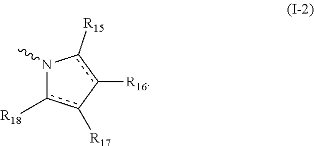

18. The tripartite tool of claim 17, wherein one of R₁₆ and R₁₇ is OH and the other is hydrogen.

19. The tripartite tool of claim 17, wherein [symbol] markings represent single bonds.

20. The tripartite tool of claim 11, wherein Rₓ is

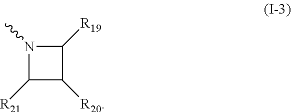

21. The compound of claim 20, wherein R₁₉ and R₂₁ are each hydrogen and R₂₀ is OH.

22. The tripartite tool of claim 11, wherein Rₓ is one of

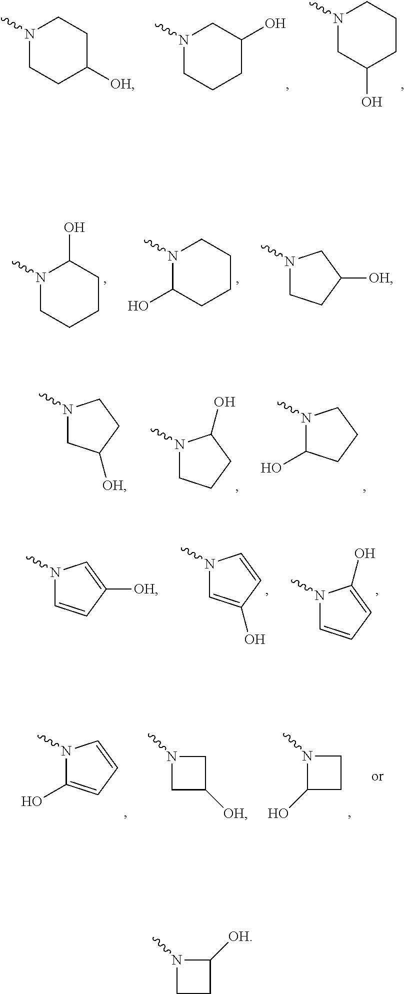

23. The tripartite tool of claim 11, wherein the compound of Formula (1) is

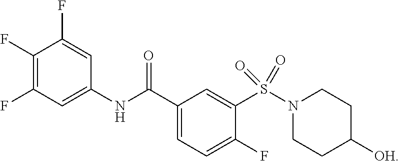 [SMILES: O=C(Nc1cc(F)c(F)c(F)c1)c1ccc(F)c(S(=O)(=O)N2CCC(O)CC2)c1]

24. The tripartite tool of claim 11, wherein the linker is 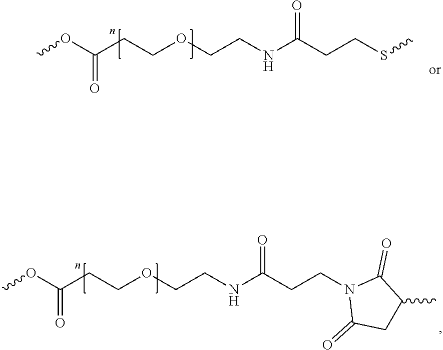
the wave-line from the sulfur or carbon atom denoting the connection to the protein, and the wave line from the oxygen atom denoting the connection to a compound of Formula (1);
wherein n is an integer from 1 to 20.

26. The tripartite tool of claim 11, wherein the tripartite tool is 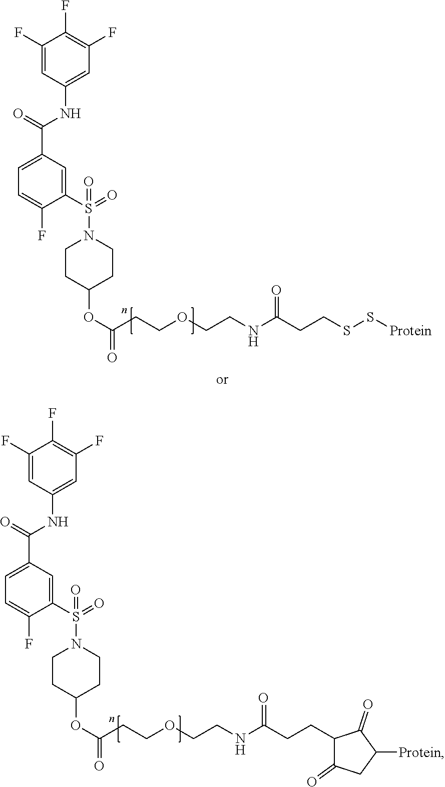
wherein n is an integer from 1 to 20.
